(19) United States
(12) Patent Application Publication
(10) Pub. No.: US 2007/0135444 A1
(43) Pub. Date: Jun. 14, 2007

(54) Treatment of neuroblastoma

(76) Inventors: Renaud Capdeville, Riedesheim (FR); Valerie Combaret, Lyon (FR); Didier Frappaz, Chaponost (FR); Alain Puisieux, Ruy (FR); Christophe Bergeron, Sainte Foy Les Lyon (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/652,417

(22) Filed: Jan. 11, 2007

Related U.S. Application Data
(63) Continuation of application No. 10/503,539, filed on Aug. 4, 2004.

(30) Foreign Application Priority Data
Feb. 7, 2002 (GB) 0202874.4

(51) Int. Cl. A61K 31/506

(52) U.S. Cl. 514/252.18

(57) ABSTRACT

4-(4-methylpiperazin-1-ylmethyl)-N-[4-methyl-3-(4-pyridin-3-yl)pyrimidin-2-ylamino)phenyl]-benzamide or a pharmaceutical acceptable salt thereof can be used in the treatment of neuroblastoma. The invention also relates to a combination of 4-(4-methylpiperazin-1-ylmethyl)-N-[4-methyl-3-(4-pyridin-3-yl)pyrimidin-2-ylamino)phenyl]-benzamide or a pharmaceutical acceptable salt thereof with one or more antineoplastic agents.

The invention relates to the use of 4-4-methylpiperazin-l-ylmethyl)-N-[4-methyl-3-(4-pyridin-3-yl)pyrimidin-2-ylamino)phenyl]-benzamide (hereinafter: “COMPOUND I”) or a pharmaceutically acceptable salt thereof for the manufacture of pharmaceutical compositions for the treatment of neuroblastoma, to the use of COMPOUND I or a pharmaceutically acceptable salt thereof in the treatment of neuroblastoma, to a method of treating warm-blooded animals including mammals, especially humans, suffering from neuroblastoma by administering to said animal in need of such a treatment an effective dose of COMPOUND I or a pharmaceutically acceptable salt thereof. The invention also relates to a combination of COMPOUND I or a pharmaceutically acceptable salt thereof with one or more other antineoplastic agents to treat warm-blooded animals including mammals, especially human and most preferably juveniles having neuroblastoma.

Neuroblastoma is a form of cancer that occurs in infants, children, and very rarely, adults. Neuroblastoma is the third most common malignant disease of infancy and 96% of cases occur before the age of 10 years. The disease is responsible for approximately 15% of all childhood cancer deaths. The neuroblastic tumor is derived from primordial neural crest cells which ultimately populate the sympathetic ganglia, adrenal medulla and other sites.

COMPOUND I is 4-4-methylpiperazin-1-ylmethyl)-N-[4-methyl-3-(4-pyridin-3-yl)pyrimidin-2-ylamino)phenyl]-benzamide having the formula I

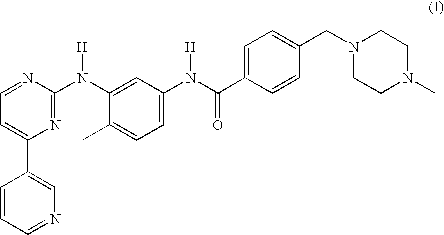

The preparation of the 4-4-methylpiperazin-1-ylmethyl)-N-[4-methyl-3-(4-pyridin-3-yl)pyrimidin-2-ylamino)phenyl]-benzamide is described in the EP-A-0 564 409.

Pharmaceutically acceptable salts of COMPOUND I are pharmaceutically acceptable acid addition salts, like for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids,.such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxy-ethane-sulfonic acid, or aromatic sulfonic acids, for example benzene-, p-toluene- or naphthalene-2-sulfonic acid.

The monomethanesulfonic acid addition salt of COMPOUND I (hereinafter “SALT I”) and a preferred crystal form thereof, e.g. the beta-crystal form, are described in PCT patent application WO99/03854 published on Jan. 28, 1999.

Surprisingly, it was found that COMPOUND I or a pharmaceutically acceptable salt thereof, e.g. SALT I, is particularly useful for the treatment of neuroblastoma.

The invention thus relates to the use of COMPOUND I or a pharmaceutically acceptable salt thereof, e.g. SALT I, as a drug against neuroblastoma. The present invention also pertains to the use of COMPOUND I or a pharmaceutically acceptable salt thereof, preferably SALT I, and most preferably the beta-crystal form of SALT I, in the manufacture of a medicament for the treatment of neuroblastoma.

The term “neuroblastoma” means any tumor deriving from nerve cells of the adrenal glands, the sympathetic nervous system ganglia of the abdomen, the sympathetic ganglia of the chest or neck or the parasympathetic ganglia in the pelvis.

Hence, the invention relates to a method of treating a warm-blooded animal having neuroblastoma comprising administering to said animal in need for such a treatment COMPOUND I or a pharmaceutically acceptable salt thereof, preferably SALT I, and most preferably the beta-crystal form of SALT I, in a quantity which is therapeutically effective against neuroblastoma.

The invention relates to a method for administering to a human subject suffering from neuroblastoma an acid addition salt and preferably the monomethanesulfonate salt of 4-(4-methylpiperazin-1-ylmethyl)-N-[4-methyl-3-4-pyridin-3-yl)pyromidin-2-ylamino)phenyl]-benzamide of the formula I and most preferably the beta-crystal form.

In a preferred embodiment of the invention, the patient to be treated is a juvenile.

SALT I is administered, however the dosages are expressed as the dose of COMPOUND I free base administered, e.g. for a 100 mg dose, 119.5 mg of SALT I being administered corresponding to 100 mg of COMPOUND I free base. For example, a dose of 400 mg of COMPOUND I as to be understood as 478 mg SALT I being administered corresponding to 400 mg of COMPOUND I free base.

Depending on age, individual condition, mode of administration, and the clinical picture in question, effective doses of COMPOUND I, for example daily doses of from 100 to 1000 mg, e.g. of 100 to 800 mg, e.g. of 100 mg to 600 mg, e.g. 400 mg, are administered to warm-blooded animals of about 70 kg bodyweight. Preferably, the warm-blooded animal is a human and most preferably a juvenile. The daily doses for a juvenile are of 100 to 900 mg/m² of body surface/ day, preferably 100 to 700 mg/m²/day, e.g. 100 to 400 mg/m² of body surface, 150 to 350 mg/m² of body surface, 340, 440, 570 or 700 mg/m² day. For patients with an inadequate response to daily doses, dose escalation or de-escalation can be safely considered and patients may be treated as long as they benefit from treatment and in the absence of limiting toxicities.

The invention relates especially to a method wherein a daily dose of 100 to 1000 mg, e.g. of 100 to 800 mg, e.g. of 200 to 600 mg, e.g. 400 mg, of COMPOUND I or a pharmaceutically acceptable salt thereof, e.g. SALT I, is administered to an adult and 100 to 900 mg/m², preferably 100 to 700 mg/m², e.g. 200 to 400 mg/m², 340, 440, 570, 700 mg/m² of body surface to a juvenile.

The invention relates also to a method for administering to a human subject suffering from neuroblastoma, COMPOUND I or a pharmaceutically acceptable salt thereof, e.g. SALT I, which comprises administering a pharmaceutically effective amount of COMPOUND I or a pharmaceutically acceptable salt thereof, e.g. SALT I, to a human subject once daily for a period exceeding 3 months.

Surprisingly, it has been found a synergistic effect of a combination as defined herein as its effect against neuroblastoma is greater than the effects that can be achieved with either type of combination partner alone, i.e. greater than the effects of a monotherapy using only one of the combination partners as defined herein. All the more surprising is the finding that the administration of COMPOUND I or a pharmaceutically acceptable salt thereof, e.g. SALT I, in combination with, at least, another antineoplastic agent, results in a surprising beneficial effect that a lower dose of the active compounds in combination can be used. This is in accordance with the desires and requirements of the patients to be treated. The person skilled in the pertinent art is fully enabled to select relevant test models to prove such beneficial effects. The pharmacological activity of a combination may, for example, be demonstrated in a clinical study or in a test procedure as essentially described hereinafter.

The invention also relates to a combination which comprises (a) COMPOUND I or a pharmaceutically acceptable salt thereof, e.g. SALT-I, and, at least, (b) another antineoplastic drug for the treatment of neuroblastoma.

The term “antineoplastic drug” used herein includes, but is not limited to aromatase inhibitors, antiestrogens, topoisomerase I inhibitors, topoisomerase II inhibitors, microtubule active agents, alkylating agents, antineoplastic antimetabolites, platin compounds, compounds decreasing the protein kinase activity and further anti-angiogenic compounds, gonadorelin agonists, anti-androgens, bisphosphonates and trastuzumab and preferably selected from the group comprising vincristine, cisplatin, doxorubicine, cyclosphosphamide, fluorouracil, tenoposide, etoposide or ifosfamide.

Suitable clinical studies are, e.g., open-label phase II, randomized, double-blind, parallel studies on patients with neuroblastoma. Such studies are, in particular, suitable to compare the effects of a monotherapy using the active ingredients and a combined therapy and to prove in particular the synergism of the active ingredients of the combination. The primary endpoints in such studies can be the effect on the progression of the disease (radiologic evaluation of tumors in regular time periods, e.g. every 8 or 12 weeks), morbidity or mortality. In a suitable study design, patients are, for example, randomized in a double-blind fashion receiving a combination of COMPOUND I or a pharmaceutically acceptable salt thereof, e.g. SALT I, and a second antineoplastic drug or a corresponding placebo. The minimum duration of such a study should be about 3 or 12 months.

In particular, if the warm-blooded animal is a human, the dosage of COMPOUND I, e.g. SALT I, is preferably of 100 mg to 1000 mg/day, e.g. of 100 mg to 800 mg/day, e.g. of 200 mg to 600 mg/day.

The suitable combination partner (b) can be, e.g. doxorubicin, cyclophosphamide, teniposide, etoposide, cisplatin or vincristine.

The suitable dose of the combination partner (b) that can be administered is determined by the person skilled in the art, e.g. 20 to 80 mg/m² of body surface/21 days of doxorubicin, 400 to 4200 mg/l²/days of cyclophosphamide, 60 to 200 mg/m²/21 days of teniposide, 150-600 mg/m²/21 days of etoposide, 30 to 300 mg/m²/21 days of cisplatin, 1 to 5 mg/m²/day of vincristine.

It is one objective of this invention to provide a pharmaceutical composition comprising a quantity, which is jointly therapeutically effective against neuroblastoma, of the combination partners. In one embodiment of the invention, the combination partners (a) and (b) of the combination are present in synergistically effective amounts and can be administered together, one after the other or separately in one combined unit dosage form or in two separate unit dosage forms. The unit dosage form may also be a fixed combination.

Preferably, the unit dosage form contains 20 to 200 mg of COMPOUND I, e.g. SALT I.

In particular, if the warm-blooded animal is a human of 70 kg body weight, the dosage of COMPOUND I, e.g. SALT I, in the combination is preferably of 100 mg to 1000 mg/day, e.g. of 100 mg to 800 mg/day, e.g. of 200 mg to 600 mg/day.

Moreover, the present invention provides a commercial package comprising as active ingredients (a) COMPOUND I or a pharmaceutically acceptable salt thereof, e.g. SALT I, and at least one compound selected from the (b) antineoplastic agents, together with instructions for simultaneous, separate or sequential use thereof in the treatment of neuroblastoma.

### EXAMPLE 1

### In vitro Effects of COMPOUND I, e.g. SALT I, Treatment on Neuroblastoma Cell Lines

Five neuroblastoma cell lines are used (IMR32, SKNAS, CLB-PE, CLB-Ma1 and CLB-Ga) to test the effect of COMPOUND I. CLB-Ma1, CLB-Ga and CLB-Pe cell lines and their culture media are disclosed in Combaret V. et al. Int. J. Cancer. 1995: 61:185-191. SKNAS and IMR32 cell lines are available commercially from SIGMA (European Collection of Cell Cultures). After division and medium change, cells from stock culture are seeded on cell plates and cultured for 18 h to allow cell growth and attachment before starting the assay. Then on the first day of the assay, COMPOUND I, e.g. SALT I, is added to the medium. Different concentrations of the drug (COMPOUND I) are used (0.1 μM, 1 μM, 5 μM, 10 μM, 20 μM and 50 μM). Cells are cultured-up to 96 h or 168 h.

³H-thymidine incorporation. DNA synthesis is evaluated by measuring ³H-thymidine incorporation. Cells are seeded in 96 well plates (1 to 2×10⁴ cells in 200 μl/well). Five replicate wells are used for each experimental condition. 3H-thymidine is added to the wells (0.5 μCi/well) and incubated with the cells for 18 h. Cells are then lysed by two freezing and thawing cycles and harvested on glass fiber filters with a semi-automatic cell harvester; the cellular ³H-thymidine uptake is determined in liquid scintillation liquid with a B scintillation counter. The ³H-thymidine incorporation is measured every 24 h.

Table 1. Growth Inhibition of Neuroblastoma Cell Lines by COMPOUND I e.g. SALT I

The table relates to the COMPOUND I concentrations for which a 50% reduction in the ³H-thymidine incorporation is obtained in comparison to the controls (no SALT I, e.g. COMPOUND I, treatment):

| Cell line | COMPOUND I concentration | |
|---|---|---|
| SKNAS | 5 | μM |
| CLB-Pe | 5 | μM |
| CLB-Ma1 | 5-10 | μM |
| CLB-Ga | 1-5 | μM |
| IMR32 | 5-10 | μM |

In response to SALT I, e.g. COMPOUND I, treatment, cell growth of the five cell lines is decreased. A 50% inhibition of cell growth is observed with different concentrations of SALT I, e.g. COMPOUND I, with different cell lines.

### EXAMPLE 2

### Clinical Trials

Plan of investigations: The present proposal will be an open label, single arm, multi-center phase II trial in patients with resistant or recurrent cancer with tumor cells expressing the c-kit protein (CD117) or the platelet-derived growth factor receptor (PDGFR) tyrosine kinase with measurable disease. Expression of these kinases will be investigated in tumor samples obtained at the time of diagnosis or from the time of recurrent disease. Tumor sections will be obtained either from paraffin blocks, or preferably from fresh tumor samples (essential particularly for reliable expression of PDGFR). Every patient with positive expression of either of the kinases will be evaluated for quantitative and qualitative evidence of disease prior to entry into the study, and if possible, no other treatment will be given concomitantly, to allow evaluation of the net effect of SALT I on tumor growth kinetics, searching for measurable evidence of response.

Medication: Patients receive SALT I at a dose corresponding to 400 mg p.o./day of COMPOUND I for an exposure period of up to 12 months provided that in the opinion of the investigator the patient is benefiting from treatment with SALT I, and in the absence of any safety concerns. SALT I may be increased to a dose corresponding to 600 mg p.o./day and then to 400 mg b.i.d of COMPOUND I if the patient is not responding. For patients experiencing Grade 2 or ¾ non-hematological or hemalotogical toxicities, SALT I will be resumed to lower doses, e.g. 300, 400 or 600 mg/daily. The administration of any other anticancer agents including chemotherapy and biologic agents is NOT, however, permitted.

Inclusion criteria: Patients with progressive neuroblastoma with residual disease or with disease resistant to chemotherapy, or children with neuroblastoma in stage IV older than 1 year regardless of response to conventional or high dose chemotherapy in view of poor prognosis on any available modality. Hence, patients failing conventional anti-cancer modalities expressing c-kit (CD 117) or PDGF-R with dismal prognosis are eligible for SALT I with a life expectancy of >3 months and measurable tumor as assessed by radiologic parameters and/or elevated tumor markers or any disease related sign or evidence of disease are eligible.

Study End points: Measurable or evaluable signs of anti-tumor effects, time to tumor progression and survival

Response Criteria

Obiective Tumor Response: complete response is defined as inability to detect evidence of disease by any of the imaging methods, or documentation of malignant cells or signs of disease by morphologic, histologic, cytogenetic findings if a disease-specific abnormality had been detected prior to therapy.

Response Duration: is defined as the time a complete response is documented to the time of relapse or last contact.

Time to progession: time to documentation of tumor progression by any of the disease-specific parameters detected prior to therapy.

Progression-free survival: is defined as the time a continuous complete response is maintained without the event of death.

Overall survival and disease-free survival: survival and disease free survival will be recorded.

Performance Status: Visit and Follow-up for Assessment of Efficacy of SALT I

| | Week | | | | | Month | | | | | |
|---|---|---|---|---|---|---|---|---|---|---|---|
| | −1 | 2 | 4 | 6 | 8 | 3 | 4 | 5 | 6 | 9 | 12 |
| Physical¹ | X | X | X | | | | | | | | |
| CBC² | X | X | X | X | X | X | X | X | X | X | X |
| C&B³ | X | X | X | | X | X | X | X | X | X | X |
| CF⁴ | X | | | | | | | | | | |
| TM⁵ (if positive) | X | X | X | | X | X | X | X | X | X | X |
| RE⁶ | X | | | X | | X | | | X | X | X |

Evaluation may be performed at more frequent intervals if desired or indicated
¹Visits include analysis and assessment of side effects and effects, physical examination and evaluation of the patient according to ECOC criteria;
²Complete Blood Count;
³Routine Chemistry and Biochemistry examinations;
⁴Coagulation Factors PT, PTT, INR;
⁵Tumor markers (depend on tumor investigated) with proper objective measurements;
⁶Response Evaluation depends on each tumor and on the specific parameters for quantitative and qualitative evaluation of disease including ultrasound examination, CT scan, etc.

Evaluation may be performed at more frequent intervals if desired or indicated

### EXAMPLE 3

### Phase II Study of SALT I in Children with Refractory Solid Tumors

Experimental design: The goal of the present clinical trial study is to estimate the response rate to SALT I in neuroblastoma recurrent solid tumors of childhood. The aim of the present study is also to correlate the SALT I response with c-kit and PDGF-R expression as determined by immunochemical staining of tumor specimens obtained at diagnosis or relapse and to describe the range of c-kit and PDGF-R expression arising in selected pediatric neuroblastomas expressing those receptors. This is a phase II study of SALT I given orally. It includes a single inter-patient dose escalation or de-escalation, using standard phase I criteria. Patients with stable or responding disease may receive the drug for a period not to exceed two years.

Rationale for dose escalation: no maximal dose tolerated was reached in the pediatric phase I trial (up to 570mg/m²/day) suggesting that further dose escalation may be feasible in children. SALT I pharmacokinetics supports single daily dosing. However, twice daily dosing can reduce side effects for administration of doses above or equal to 800 mg per day in adult and pediatric trials.

Patient eligibility: Patients must be below 30 years of age at the time of entry on study. Patients have had histologic verification of neuroblastoma at original diagnosis and the tumor must be recurrent or refractory to standard treatment. Patients must have measurable disease documented by CT or MRI imaging and have relapsed or become refractory to conventional therapy. The presence of at least one lesion that can be accurately measured in at least one dimension is required. Administration schedule: SALT I is administered orally in two divided doses daily. SALT I is given once daily for doses 500 mg/day or less and divided b.i.d doses for 600 mg/day and above. If an odd number of hundreds (e.g. 700 mg/day) occurs the larger doses is given in the evening (i.e. 300 mg/day in the morning and 400 mg/day in the evening). In the absence of clinical and radiological progressive disease, patients should continue to receive SALT I.

Inter-Patient escalation: The initial dose level is 570 mg/n² of body surface/day with a subsequent group of patients treated at 700 mg/m²/day. If the maximal dose tolerated is exceed at the first dose level then the subsequent cohort of patients will be treated at 440 mg/m²/day. If the maximal dose tolerated is exceed at the dose level of 700 mg/m²/day then the subsequent cohort of patients will be treated at 570 mg/m² of skin/day. Dose escalation is safely considered. If the maximal tolerated dose is not being exceeded the dose will remain at 700 mg/m²/day for. subsequent patient entry into the study.

Biology: tissue blocks of tumors from diagnosis or relapse prior to enrollment is used for immunohistochemical evaluation for c-kit and PDGF receptor expression and for c-kit and PDGF receptor mutation analysis.

Evaluabilityfor response: Response rates and confidence intervals are constructed according to the method of Chang and O'Brien (Chang M N et al., Biometrics (1987) 43:864-74 Chang M N and O'Brien P C (1987) Controlled Clinical Trials 7:18-26). This study uses the (RECIST) Response Evaluation Criteria in Solid Tumors from the National Cancer Institute (NCI). Serial-measurement of lesions are done with CT or MRI. The sum of the longest diameter for all target lesions is calculated and reported as the disease measurement. The disease burden is then reported as complete response, partial response, progressive disease or stable disease. Toxicity is also evaluated and reported.

### EXAMPLE 4

### Tablets with 4-[(4-methyl-1-piperazin-1-ylmethyl)-N-[4-methyl-3-[[4-(3-pryidinyl-2-pyrimidinyl]amino]-phenyl]benzamide methanesulfonate, β-crystal form

Tablets containing 119.5 mg of the compound named in the title (SALT I) and corresponding to 100 mg of COMPOUND I are usually prepared in the following composition:

| | | |
|---|---|---|
| Active ingredient | 119.5 | mg |
| Crystalline lactose | 240 | mg |
| Avicel | 80 | mg |
| PVPPXL | 20 | mg |
| Aerosil | 2 | mg |
| Magnesium stearate | 5 | mg |
| | 466.5 | mg |

Preparation: The active substance is mixed with carrier materials and compressed on a tableting machine (Korsch EKO, punch diameter 10 mm).

Avicel is microcrystalline cellulose (FMC, Philadelphia, USA).

PVPPXL is polyvinylpolypyrrolidone, cross-linked (BASF, Germany).

Aerosil is silicon dioxide (Degussa, Germany).

### EXAMPLE 5

### Capsules with 4-[(4-methyl-1-pipierazin-1-ylmethyl)-N-[4-methyl-3-[[4-(3-pyridinyl)-2-pyrimidinyl]amino]phenyl]benzamide methanesulfonate, β-crystal form

Capsules containing 119.5 mg of the compound named in the title (=SALT I) corresponding to 100 mg of COMPOUND I are prepared in the following composition:

| | | |
|---|---|---|
| SALT I | 119.5 | mg |
| Avicel | 200 | mg |
| PVPPXL | 15 | mg |
| Aerosil | 2 | mg |
| Magnesium stearate | 1.5 | mg |
| | 338 | mg |

The capsules are prepared by mixing the components and filling the mixture into hard gelatin capsules, size 1.

1. Use of 4-(4-methylpiperazin-1-ylmethyl)-N-[4-methyl-3-(4-pyridin-3-yl)pyrimidin-2-ylamino)phenyl]-benzamide of the formula I 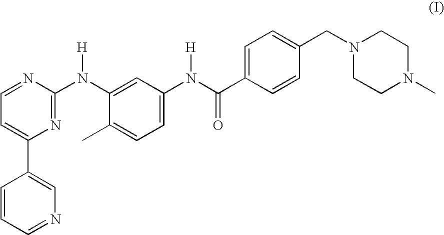
or a pharmaceutically acceptable salt thereof for the manufacture of a medicament for the treatment of neuroblastoma.

2. Use of 4-(4-methylpiperazin-1-ylmethyl)-N-[4-methyl-3-(4-pyridin-3-yl)pyrimidin-2-ylamino)phenyl]-benzamide of the formula I or a pharmaceutically acceptable salt thereof in the treatment of neuroblastoma.

3. The use according to claim 1 wherein 4-(4-methylpiperazin-1-ylmethyl)-N-[4-methyl-3-(4-pyridin-3-yl)pyrimidin-2-ylamino)phenyl]-benzamide of the formula I is in the form of the monomethanesulfonate salt thereof.

4. A method of treating a human suffering from neuroblastoma which comprises administering to said human in need of such a treatment a dose, effective against neuroblastoma, of 4-(4-methylpiperazin-1-ylmethyl)-N-[4-methyl-3-(4-pyridin-3-yl)pyrimidin-2-ylamino)phenyl]-benzamide of the formula I or a pharmaceutically acceptable salt thereof.

5. The method according to claim 4 wherein a daily dose of 4-(4-methylpiperazin-1-ylmethyl)-N-[4-methyl-3-(4-pyridin-3-yl)pyrimidin-2-ylamino)phenyl]-benzamide of the formula I of 100 to 900 mg/m² of body surface is administered to a juvenile.

6. The method according to claim 4 wherein a daily dose of 4-(4-methylpiperazin-1-ylmethyl)-N-[4-methyl-3-(4-pyridin-3-yl)pyrimidin-2-ylamino)phenyl]-benzamide of the formula I of 100 to 700 mg/m² of body surface is administered to a juvenile.

7. The method according to claim 4 wherein a daily dose of 100 to 1000 mg of 4-(4-methylpiperazin-1-ylmethyl)-N-[4-methyl-3-(4-pyridin-3-yl)pyrimidin-2-yl-amino)phenyl]-benzamide of the formula I is administered to an adult.

8. The method according to claim 4 wherein the monomethanesulfonate salt of 4-(4-methylpiperazin-1-ylmethyl)-N-[4-methyl-3-(4-pyridin-3-yl)pyrimidin-2-ylamino)phenyl]-benzamide of the formula I is administered.

9. A method of treating humans suffering from neuroblastoma which comprises administering to a said mammal in need of such treatment a pharmaceutical composition comprising
(a) a dose, effective against neuroblastoma, of 4-(4-methylpiperazin-1-ylmethyl)-N-[4-methyl-3-(4-pyridin-3-yl)pyrimidin-2-ylamino)phenyl]-benzamide of the formula I or a pharmaceutically acceptable salt thereof and
(b) a therapeutically effective amount of a second antineoplastic agent.

10. A combination which comprises (a) a dose, effective against neuroblastoma, of 4-(4-methylpiperazin-1-ylmethyl)-N-[4-methyl-3-(4-pyridin-3-yl)pyrimidin-2-ylamino)phenyl]-benzamide of the formula I or a pharmaceutically acceptable salt thereof and
(b) a therapeutically effective amount of a second antineoplastic agent.

11. A method or a combination according to claim 9 wherein the second antineoplastic drug (b) is selected from the group consisting of vincristine, cisplatin, doxorubicine, cyclosphosphamide, fluorouracil, etoposide and ifosfamide.

12. A combination according to claim 10 wherein the combination partners are present in synergistically effective amounts.
